(19) United States
(12) Patent Application Publication
(10) Pub. No.: US 2008/0274446 A1
(43) Pub. Date: Nov. 6, 2008

(54) Cryopreserved human neuronal cultures

(75) Inventors: Steven Stice, Athens, GA (US); David Machacek, Athens, GA (US)

Correspondence Address: COLEMAN SUDOL SAPONE, P.C., 714 COLORADO AVENUE, BRIDGE PORT, CT, 06605-1601, US

(73) Assignee: THE UNIVERSITY OF GEORGIA RESEARCH FOUNDATION, INC., Athens, GA (US)

(21) Appl. No.: 12/150,818

(22) Filed: May 1, 2008

Related U.S. Application Data
(60) Provisional application No. 60/927,851, filed on May 4, 2007.

(51) Int. Cl. A01N 1/02; C12N 5/08

(52) U.S. Cl. 435/1.3; 435/368

(57) ABSTRACT

Cryopreserved cultures of post-mitotic neuronal or neural-like cells are provided having a viability after thaw of greater than 10%, typically greater than 50%. Once thawed, the cells are capable of further differentiation. In one embodiment, less than 20% of the cryopreserved cells are self-proliferating cells. The cells can be provided in a kit including a container of the cryopreserved neuronal or neural-like cells, optionally including additional cell culture reagents and materials. Method for preparing the cryopreserved neuronal or neural-like cells derived from embryonic stem cells, preferably human embryonic stem cells, are also provided.

### RELATED APPLICATIONS

This application claims priority from U.S. provisional application Ser. No. 60/927,851, filed May 4, 2007, which is incorporated by reference in its entirety herein.

### TECHNICAL FIELD

Aspects of the invention are generally directed to cell cultures and methods of preserving them, in particular, to neuronal cell cultures.

### BACKGROUND OF THE INVENTION

Stem cell research has been heralded as having the potential to provided cures to many different forms of disease or injury, particularly neurodegenerative diseases. Embryonic stem (ES) cells can theoretically provide unlimited amounts of every cell type. ES cells have provided a valuable and convenient source of neural cells (Gottlieb and Huettner, Cells Tissues Organs. 165(3-4):165-72 (1999)). Although it is possible to differentiate ES cells in vitro into more mature cell types, it remains difficult to direct the differentiation of an entire population of ES cells into progenitors of a single tissue type. Progenitor cells are highly proliferative immature cells.

Research on the cell fate specification in the central nervous system is of enormous interest given the therapeutic potential in neuronal repair strategies. Human ES (hES) cells are believed to be a promising future source of cells for cell replacement therapy. For neurological disorders, hES cells could provide an virtually infinite source of self-renewing cells that can be persuaded to differentiate into specific types of neural cells, including dopaminergic cells (Lindall, O. et al. Nat. Med. S42-50 (2004); Ben-Hur, T. Future Neurol. 1:227-236 (2006)). However, the molecular mechanisms that govern development of culture hES cells into specific neural cells are not fully understood.

Some researchers are interested in using human neural progenitors for research purposes because these cells are highly proliferative. However, neural progenitor cells take time to develop into more mature neural phenotypes which contain post-mitotic cells. Inducing neural progenitor cells to differentiate into specific neural cells is challenging. Post-mitotic neural cells are more useful in research because they are more similar to the neural cells in a patient. A source of more mature neural or neural-like cells that can be obtained easily and reliably would be advantageous, particularly in drug discovery for the treatment of neurological disorders.

Cryopreservation of neuronal cells could provide a source of neural cells. However, cryopreservation of neural cells is problematic and conventional methods of cryopreservation of neurons typically have a survival rate of about 10% or less after thaw. The low survival rate is due in part because cryopreservation typically destroys structural components of the neuron such as the axon on other outgrowths and cytoskeletal components. Thus, cryopreservation techniques for neurons that have greater than 10% survivability rates after thaw are desirable.

Therefore, it is an object of the invention to provide a method of cryopreserving post-mitotic neural cells derived from ES cells wherein a high percentage can be thawed and induced to further differentiate.

Another object of the invention provides media and a process that allows for the cryopreservation and survival of differentiated human neurons derived from stem cells.

### SUMMARY OF THE INVENTION

It has been discovered that post-mitotic neuronal or neural-like cells can be cryopreserved and have greater than 10%, typically greater than 30%, even more typically greater than 50%, survivability after thawing. The very high percent viability following thawing allows the cells to be shipped and stored for use when needed, rather than being production driven. This is a significant commercial advance. The post-mitotic neuronal or neural-like cells are derived from primate embryonic stem cells, preferably human embryonic stem cells, by induction. In one embodiment, the cryopreserved cells contain less than 20% self-proliferating neuronal or neural-like cells. The cryopreserved cells are not fully mature, express β-tubulin III, and are able to continue differentiating after thawing. After the cultures are thawed, neurite outgrowths can be seen, for example within about 10 to about 14 hours, preferably about 12 hours.

The cells are typically stored in a container suitable for tissue culture. A kit optionally includes a cell adhesion promoter, defined culture media, media supplements, and tissue culture-ware. The media can be provided in powder or liquid form. The kit also includes written instructions for thawing and culturing the cryopreserved cells.

Methods for deriving the neuronal or neural-like cells from embryonic stem cells are provided as well as methods for cryopreserving the cells, methods for thawing the cells, and methods for cultivating the cells. The method utilizes a defined culture protocol and a defined culture medium suitable for culturing the cryopreserved neuronal or neural-like cells for about 10 to about 20 days until the cells begin to express beta tubulin 3. The cells are then frozen.

The cryopreserved cells can be used as research tools for investigating the differentiation and development of neurons. The cells can also be used to screen for potentially therapeutic agents for treating damaged or diseased nervous tissue.

### DETAILED DESCRIPTION OF THE INVENTION

### I. Definitions

The following definitions are provided to clarify the various embodiments of the invention. The definitions are used in a manner keeping with their common meaning as understood by one of ordinary skill in the art.

The term “administration” or “administering” refers to the process by which neural or neural-like cells derived from ES cells are delivered to a patient for treatment purposes. Neural or neural-like cells may be administered a number of ways including parenteral (such term referring to intravenous and intraarterial as well as other appropriate parenteral routes), intrathecal, intraventricular, intraparenchymal (including into the spinal cord, brainstem or motor cortex), intracistemal, intracranial, intrastriatal, and intranigral, among others which term allows neural or neural-like cells to migrate to the cite where needed. Administration will often depend upon the disease or condition treated and may preferably be via a parenteral route, for example, intravenously, by administration into the cerebral spinal fluid or by direct administration into the affected tissue in the brain. For example, in the case of Alzheimer's disease, Huntington's disease and Parkinson's disease, the preferred route of administration will be a transplant directly into the striatum (caudate cutamen) or directly into the substantia nigra (Parkinson's disease). In the case of amyotrophic lateral sclerosis (Lou Gehrig's disease) and multiple sclerosis, the preferred administration is through the cerebrospinal fluid. In the case of lysosomal storage disease, the preferred route of administration is via an intravenous route or through the cerebrospinal fluid. In the case of stroke, the preferred route of administration will depend upon where the stroke is, but will often be directly into the affected tissue (which may be readily determined using MRI or other imaging techniques).

The term “cell adhesion promoter” refers to a substance that promotes cell adhesion to a substrate. Exemplary cell adhesion promoters include, but are not limited to laminin, polyornithine, and combinations thereof.

The term “cell medium” or “cell media” is used to describe a cellular growth medium in which embryonic stem cells, neuroprogenitor cells and/or neural, neural-like or neuronal cells are grown. Cellular media are well known in the art and comprise at least a minimum essential medium plus optional agents such as growth factors, including fibroblast growth factor, preferably basic fibroblast growth factor (bFGF), leukemia inhibition factor (LIF), glucose, non-essential amino acids, glutamine, insulin, transferrin, beta mercaptoethanol, and other agents well known in the art. Preferred media include commercially available media such as DMEM/F12 (1:1) or neurobasal media, each of which may be supplemented with any one or more of L-glutamine, knockout serum replacement (KSR), fetal bovine serum (FBS), non-essential amino acids, leukemia inhibitory factor (LIF), beta-mercaptoethanol, basic fibroblast growth factor (bFGF) and an antibiotic, B27 medium supplement and/or N2 medium supplement. Useful cell media are commercially available and can be supplemented with commercially available components, available from Invitrogen Corp. (GIBCO) and Biological Industries, Beth HaEmek, Israel, among numerous other commercial sources. In preferred embodiments at least one differentiation agent is added to the cell media in which a stem cell or neuroprogenitor cell is grown in order to promote differentiation of the stem cells into neuroprogenitor cells and the neuroprogenitor cells into motor neuron cells. One of ordinary skill in the art will be able to readily modify the cell media to produce neuroprogenitor or most-mitotic neural cells described herein.

The term “embryonic stem cell” refers to pluripotent cells, preferably of primates, including humans, which are isolated from the blastocyst stage embryo. Human embryonic stem cell refers to a stem cell from a human and is preferably used in aspects which relate to human therapy or diagnosis. The following phenotype markers are expressed by human embryonic stem cells: SSEA-3, SSEA-4, TRA-1-60, TRA-1-81, GCTM-2, TG343, TG30, CD9, Alkaline phosphatase, Oct 4, Nanog, TRex 1, Sox2, TERT and Vimentin. See Ginis, et al., Dev. Biol, 269(2), 360-380 (2004); Draper, et al., J. Anat., 200(Pt. 3), 249-258, (2002); Carpenter, et al., Cloning Stem Cells, 5(1), 79-88 (2003); Cooper, et al., J Anat., 200(Pt.3), 259-265 (2002); Oka, et al., Mol. Biol. Cell, 13(4), 1274-81 (2002); and Carpenter, et al., Dev. Dyn., 0229(2), 243-258 (2004). While any human stem cell can be used in the present methods to produce human neuroprogenitor cells and postmitotic neuronal cells, preferred human embryonic stem cells for use in the present invention include stem cells from the WA09 cell line as well as numerous other available stem cell lines.

The terms “grafting” and “transplanting” and “graft” and “transplantation” refer to the process by which neural or neural-like cells derived from ES cells are delivered to the site within the nervous system where the cells are intended to exhibit a favorable effect, such as repairing damage to a patient's central nervous system, treating a neurodegenerative disease or treating the effects of nerve damage caused by stroke, cardiovascular disease, a heart attack or physical injury or trauma or genetic damage or environmental insult to the brain and/or spinal cord, caused by, for example, an accident or other activity. Neural or neural-like cells derived from ES may also be delivered in a remote area of the body by any mode of administration as described above, relying on cellular migration to the appropriate area in the central nervous system to effect transplantation.

The term “neural cell” or “neuronal cell” or “neural-like cell” refers to a cell that expresses one or more neural markers selected from the group consisting of, Nfh, Eno2, Dat, Ngf, Hoxc6, S100β, GFAP, TapaI, Mag, Mobp, Omg, MAP2, β-TubIII, nestin, Sox2, Hu, PSNCAM, HB9, Islet 1/2, ChAT, Olig2, oligo 04/GALC, A2B5/AD4, Vimentin, PDGFRα and MBP. Cells expressing any one or any combination of these markers can be obtained from neuroprogenitor cells derived from embryonic stem cells.

The term “post-mitotic neural cell” refers to a neural cell that is non-replicating. The term “neuroprogenitor cells” or “neuroepithelial stem cells” refers to cells which are the earliest multipotent neural stem cells. These are self renewing cells that can differentiate into neurons, oligodendrocytes and astrocytes. Neuroprogenitor cells (NP) or (NEP) may be further delineated into “early neuroprogenitor cells” and “late neuroprogenitor cells”. Early neuroprogenitor cells are neuroprogenitor cells which are freshly isolated without further propagation. Late neuroprogenitor cells are neuroprogenitor cells which have been propagated for at least about three months. Neuroprogenitor cells express markers associated with the earliest multipotent neural stem cells, including Nestin, a neural intermediate filament protein, Musashi-1, a neural RNA binding protein, as well as Sox1, Sox2 and Sox3, but these cells do not express, or express very low levels of further differentiation markers such as PSNCAM, MAPH, or other late stage neuronal or glial lineage markers such as A2B5/4D4, GFAP/CD44, RC1/S100/Vimentin, Sox10/NG2/PDGFRα, 04/GALC, PLP-DM20/CNP/MBP. The expression of Sox1, Sox2 and Sox3 in the absence of Oct4 is evidence of the existence of a neuroprogenitor cell.

Neuronal-Like Cell Cultures

In one embodiment, neural or neural-like cell cultures are derived from primate stem cells, preferably human embryonic stem cells via an induction process. Upon reaching a point of maturity where the resulting cultures demonstrate morphology (extension of neurites) and protein expression consistent with predominately post-mitotic neural cultures (increased β-tubulin III), the cells are cryopreserved. After the cultures are thawed, neurite outgrowths can be seen, for example within about 10 to about 14 hours, preferably about 12 hours.

One embodiment provides cryopreserved neuronal or neural-like cells dervied from ES cells having a percent survival after thawing of greater than 10%, typically about 50% to about 90% survival rate, more typically about 60% to 80% survival rate after thawing. The neuronal or neural-like cells are preferably derived from human ES cells.

Another embodiment provides cyropreserved neuronal or neural-like cells derived from hES cells wherein greater than 20% of the cells are post-mitotic, and optionally have a survival rate of about 50 to about 80% upon thawing.

Yet another embodiment provides a cryopreserved culture of post-mitotic neuronal or neural-like cells derived from neural progenitor cells, wherein the post-mitotic neuronal or neural-like cells express β-tubilin III and have survival rate of greater than 50% upon thawing.

One embodiment provides a cryopreserved culture of neuronal or neural-like cells expressing β-tubulin III in greater amounts than neuroprogenitor cells and nestin in

### III. Methods of Manufacturing Neuronal-Like Cell Cultures

A. Production of NeuroProgenitor Cells from ESCs

Neural progenitor cells can be derived from ES cells using defined cell culture media and certain growth factors. Human embryonic stem (hES) cells can be obtained from NIH registered cultures, for example H9. Neuroprogenitor cells can be induced from hES cells according to the methods described in U.S. Patent Application Publication No. 20060073587 A1 to Stice et al. Briefly, hES cells are passaged onto feeder cells and allowed to proliferate in defined derivation culture medium for about 5 to 10, preferably 7 days. The most commonly used feeder cells in human ES cell culture are fibroblasts isolated from 13.5 days mouse embryos. One of ordinary skill in the art will recognize that other suitable feeder cells can be used.

An exemplary derivation culture medium includes Dulbecco's modified Eagle's medium (DMEM/F12; GIBCO) supplemented with 15% serum and 5% knockout serum replacement (KSR; GIBCO) 2 mM L-glutamine, 0.1 mM minimal essential medium nonessential amino acids, 50 U/ml penicillin, and 50 μg/ml streptomycin, 4 ng/ml basic fibroblast growth factor (bFGF; SIGMA-Aldrich), and 10 ng/ml leukemia inhibitory factor (LIF; Chemicon), with the use of penicllin and/or streptomycin being optional.

After culture in the derivation medium is completed, the derivation medium is replaced with a second defined medium. An exemplary second defined medium includes DMEM/F12-based medium supplemented with N2 (Gibco), 2 mM L-glutamine, pencillin/streptomycin and 4 ng/ml bFGF with penicllin and strepmycin being optional. The adherent cells are cultured in the new culture medium for about 5 to 10 days, preferably about 7 days. Subsequently, the adherent hES cells are cultured without feeder cells for about 2 to 5 days, preferably about 3 days in the same cell culture medium.

After culturing in the absence of feeder lays for about 2 to 5 days, the adherent cells are isolated and cultured in a third medium. An exemplary third culture medium contains 96% Neural Basal Media (GIBCO) supplemented with 1X Penicillin/streptomycin, 2 mM L-glutamine, 1X B27, 10 ng/ml of LIF and 20 ng/ml of bFGF, with penicillin, streptomycin, and LIF being optional components. The adherent cells are cultured on coated culture plates.

The culture plates are preferably coated with an adhesion-promoting substance, for example laminin, polyornithine, or a combination thereof. The cells may be maintained in culture indefinitely. Poly-L-ornithine and laminin provide a preferred matrix for adhesion and growth of the cells. Generally, stock solutions of poly-L-ornithine (10 mg/mL) are prepared by dissolving poly-L-ornithine in sterile water. The stock solution should be stored at −20° C. or −80° C. The poly-L-ornithine is diluted with water from the stock concentration (10 mg/mL) to yield 20 μg/mL for polystyrene plates or 50 μg/mL for glass plates. Poly-L-ornithine solution is added to cover the whole surface of the tissue culture-ware and incubated in a humidified 37° C. incubator for at least one hour. Remaining poly-L-ornithine solution is removed and the plates are rinsed once with sterile water.

Laminin is diluted to a final concentration of 5 μg/mL for both glass and polystyrene tissue culture-wares. The plates are incubated in a humidified 37° C. incubator for at least 1 hour. The coated plates or flasks can be stored in the laminin solution at 2-8° C. for 3 weeks or at −20° C. for 6-8 months.

The culture of cells typically includes greater than about 85%, typically greater than about 90% neuroprogenitor cells. The neuroprogenitor cells express nestin and do not express β-tubulin III or do not express detectable amounts of β-tubulin III.

B. Induction of NeuroProgenitor Cells to Form Neural-Like Cell Cultures.

Cultures are grown until confluent, and the medium is replaced with an induction medium without bFGF for about 2 to 5 days, preferably about 3 days. In one embodiment, the induction medium includes Neural Basal Media (GIBCO) supplemented with 1X Penicillin/streptomycin, 2 mM L-glutamine, 1X B27, 10 ng/ml of LIF. Cultures are allowed to further differentiate for 2-3 weeks with changes in media every 3-4 days. As the cultures differentiated into neural, neural-like or neuronal-like cells, the expression of nestin decreases and the expression of β-tubulin III increases indicating the cells are becoming more mature and less proliferative. Expression of nestin and β-tubulin III can be monitored using conventional techniques include for example Polymerase Chain Reactions. In one embodiment, cells are ready for cryopreservation as nestin expression decreases and β-tubulin III expression increases.

Generally, 20% or less of the hES derived neural-like cells are able to continue through mitosis. Thus, the majority of the cells are post-mitotic neural-like cells.

C. Cryopreservation of Cells

Cells derived from hES and expressing β-tubulin III can be cryopreserved. Typically, cells are harvested for cryopreservation after a majority of the cells begin to express detectable levels of β-tubulin III. Alternatively, the cells are harvested when expression of nestin decreases and the expression of β-tubulin III increases compared to neuroprogenitor cells. In one embodiment cells are harvested with nestin expression in a majority of the cells decreases by about 10% or more compared to neuroprogenitor cells or is undetectable. Still another embodiment provides harvesting the cells for cryopreservation when expression of β-tubulin III increases in a majority of the cells by about 10% or more compared to neuroprogenitor cells.

To dislodge the cultures from the plate and to gently separate the cells from each other, cultures are broken up enzymatically, for example using dispase (GIBCO). At the time of harvest equal volume of dispase 1 g/L (in PBS) is added to the cultures. Cells are observed visually through a microscope until they could be seen lifting from the plates. Occasional vibration can be applied to the dish to assist with this process. When all the cells were free from the dish, an equal volume of FBS is added to the culture to neutralize dispase activity. Cells are not forced into single cell suspensions, but remained in multicellular clumps at this point. The cells are aliquoted, for example into a falcon tube and spun in a centrifuge at 100 g. The supernatant is discarded and the pellet is gently resuspended in 10 mls of PBS followed by an additional centrifugation for 4 minutes at 100 g. Supernatant is discarded and cells were resuspended in induction medium. An estimate of cell number is determined based upon packed cell volume. Additional dilutions are added to obtain 2× the targeted number of cells per vial. Equal volume of induction medium is mixed with DMSO (20%) and added to the cells dropwise for a final concentration of 10% DMSO. Vials were then brought to −80 at 1° C. per minute and placed into liquid nitrogen for long-term storage.

D. Thawing Cryopreserved Cells

To thaw cryopreserved cells the cryopreserved cells are incubated in a 37° C. water bath until the cells are completely thawed. Maximum cell viability is dependent on the rapid and complete thawing of frozen cells. The cells should not be vortexed. As soon as the cells are completely thawed, the outside of the vial is disinfected with 70% ethanol. Cells are transferred using a pipette to culture tube and gently resuspended in induction medium. The cells are centrifuged and the supernatant discarded. The cells are suspended in induction medium and plated onto a poly-L-ornithine and laminin-coated tissue culture plates. Cells are then incubated at 37° C. in a 5% CO₂ humidified incubator.

### IV. Methods of Using Neuronal-Like Cell Cultures

A. Research Tools

Neural or neural-like cells can be used to study neuronal differentiation and development. Cryopreserved post-mitotic cells can be easily thawed and cultured to facilitate consistency in research. The thawed cells can be allowed to further differentiate depending on the culture conditions. Additional methods for fully differentiating the neural or neural-like cells derived from ES cells can be developed. Fully differentiated cells can be further used in transplant or grafting procedures to repair damaged or missing nervous system tissue. Methods for treating spinal damage and neurodegenerative disorders using the neural or neural-like cells derived from ES can be evaluated.

B. Drug Discovery

Another embodiment provides methods of screening compounds for their ability modulate the biological activity of neural or neural-like cells derived from ES cells. A test compound can be added to a culture of neural or neural-like cells derived from ES cells, and the neural or neural-like cells can be assayed for a change in phenotype. A change in phenotype includes, but is not limited to changes in shape, morphology, gene expression, protein synthesis, or structural changes. The changes in the cells contacted with the test compound can be compared to cells that were not contacted with the test compound. Exemplary test compounds are compounds believed to induce synaptic formation between cells, induce axonal outgrowth, induce secretion of neurotransmitters, prolong cell survival, repair cellular damage, or a combination thereof.

The derived neural or neural-like cells can optionally be stably or transiently transfected using conventional techniques which are well known and used in the art. For example, a transgenic neural or neural-like cell having an expression vector can be generated by introducing the expression vector into the cell. The introduction of DNA into a cell or a host cell is well known technology in the field of molecular biology and is described, for example, in Sambrook et al., Molecular Cloning 3rd Ed. (2001). Methods of transfection of cells include calcium phosphate precipitation, liposome mediated transfection, DEAE dextran mediated transfection, electroporation, ballistic bombardment, and the like. Alternatively, cells may be simply transfected with the disclosed expression vector using conventional technology described in the references and examples provided herein.

The neural or neural-like cell can be examined using any of a number of different physiologic assays. Alternatively, molecular analysis may be performed, for example, looking at protein expression, MRNA expression (including differential display of whole cell or polyA RNA) and others.

C. Administration of Cells for Transplantation

The disclosed neural or neural-like cells can be administered and dosed according conventional medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

The disclosed neural or neural-like cells an be administered in various ways as would be appropriate to implant in the central nervous system, including but not limited to parenteral, including intravenous and intraarterial administration, intrathecal administration, intraventricular administration, intraparenchymal, intracranial, intracistemal, intrastriatal, and intranigral administration. In addition, all of these routes of administration may be used to effect transplantation of neural cells derived from ES cells.

Methods of treating a patient for a neurodegenerative disease or brain and/or spinal cord damage caused by, for example, physical injury or by ischemia caused by, a stroke, heart attack or cardiovascular disease include administering neural cells to the patient in an amount sufficient to effect a neuronal transplantation. One of ordinary skill may readily recognize that one may use treated (i.e., cells exposed to at least one differentiation agent) or post-mitotic neural cells for such methods.

Pharmaceutical compositions including effective amounts of neural or neural-like cells derived from ES cells as describe above are provided. These compositions include an effective number of neural or neural-like cells derived from ES cells, optionally in combination with a pharmaceutically acceptable carrier, additive or excipient. In certain aspects, cells are administered to the patient in need of a transplant in sterile saline. In other aspects, the cells are administered in Hanks Balanced Salt Solution (HBSS) or Isolyte S, pH 7.4. Other approaches may also be used, including the use of serum free cellular media. Such compositions, therefore, include effective amounts or numbers of neural or neural-like cells in sterile saline. These may be obtained directly by using cryopreserved neural or neural-like cells derived from ES cells.

### V. Kits

One embodiment provides a kit including the cryopreserved neuronal or neural-like cells. The cells are typically stored in a container suitable for tissue culture. The kit optionally includes a cell adhesion promoter, defined culture media, media supplements, and tissue culture-ware. The media can be provided in powder or liquid form. The kit also includes written instructions for thawing and culturing the cryopreserved cells.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

We claim:

1. A composition comprising neuronal or neural-like primate cells derived from primate embryonic stem cells, cryogenically preserved under conditions wherein greater than about 10% of the cells survive after thawing.

2. The composition of claim 1 wherein greater than 50% of the cells survive after thawing.

3. The composition of claim 1, wherein less than about 20% of the neuronal-like cells undergo continued mitosis after thawing.

4. The composition of claim 1, wherein the neuronal or neural-like cells express β-tubulin III.

5. The composition of claim 1, wherein the neuronal-like primate cells are human neuronal or neural-like cells.

6. The composition of claim 1, wherein in expression of nestin is decreased compared to neuroprogenitor cells.

7. The composition of claim 1, wherein the expression of beta-tubulin III is increased compared to neuroprogenitor cells.

8. The composition of claim 1, wherein the neuronal or neural-like cells develop neurites after about 10 to about 14 hours.

9. The composition of claim 1 wherein said neuronal or neural-like cells are adherent cells.

10. The composition of claim 1 wherein said neuronal or neural-like cells are non-adherent cells.

11. A method of producing a frozen culture of neuronal or neural-like cells comprising
culturing neuroprogenitor cells in defined serum-free culture medium not containing bFGFon a substrate coated with an cell adhesion promoter for about 10 to about 20 days until the cells begin to express beta tubulin 3;
harvesting the cells; and
cryopreserving the cells.

12. The method of claim 11, wherein greater than 20% of the harvested cells are post-mitotic cells.

13. The method of claim 11 wherein the cells are capable of forming neurites within 10 to 12 hours after thawing.

14. The method of claim 11, wherein the harvested cells continue to differentiate after thawing.

15. The method of claim 11 wherein the adhesion promoter is laminin or another extracellular matrix protein or polyorthinine, or combination thereof.

16. A cryopreserved culture of post-mitotic neuronal or neural-like cells wherein the post-mitotic neuronal or neural-like cells express β-tubulin III and have a survival rate greater than 10% after thaw.

17. The cryopreserved culture of post-mitotic neuronal or neural-like cells of claim 16, wherein the post-mitotic neuronal or neural-like cells form neurites within about 10-14 hours after thaw.

18. The cryopreserved culture of post-mitotic neuronal or neural-like cells of claim 16, wherein the cells are derived from embryonic stem cells.

19. The cryopreserved culture of post-mitotic neuronal or neural-like cells of claim 18, wherein the cells are derived from human embryonic stem cells.
